(19) United States
(12) Patent Application Publication
(10) Pub. No.: US 2009/0035318 A1
(43) Pub. Date: Feb. 5, 2009

(54) METHOD AND COMPOSITION FOR INCREASING THE ENGRAFTMENT EFFICIENCY OF STEM CELLS

(75) Inventor: Connie Eaves, Vancouver (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: BRITISH COLUMBIA CANCER AGENCY BRANCH, Vancouver, BC (CA)

(21) Appl. No.: 12/088,342

(22) PCT Filed: Sep. 27, 2006
(86) PCT No.: PCT/CA2006/001592

§ 371 Date: Mar. 27, 2008

Related U.S. Application Data
(60) Provisional application No. 60/721,549, filed on Sep. 29, 2005.

(51) Int. Cl. A61K 39/395; A61K 35/12; A61P 43/00

(52) U.S. Cl. 424/158.1; 424/93.7

(57) ABSTRACT

A method is described for increasing the engraftment efficiency of S/G₂/M phase stem cells, which involves treating a recipient with the stromal cell-derived factor-1 (SDF-1) antagonist SDF-1G2 prior to delivery of the cells to said recipient. Further, a method of transplanting proliferating or S/G₂/M phase stem cells is described, comprising the steps of: (a) obtaining stem cells; (b) inducing stem cells ex vivo to proliferate or enter S/G₂/M phase; (c) treating the recipient with the SDF-1 antagonist SDF-1G2; and (d) providing the stem cells to the recipient.

### CROSS REFERENCE TO RELATED APPLICATIONS

This application claims the benefit of priority of U.S. Provisional Patent Application No. 60/721,549 filed Sep. 29, 2005, which is incorporated herein by reference.

### FIELD OF THE INVENTION

The present invention relates generally to methods and compositions for modulating engraftment of proliferating or S/G₂/M phase stem cells.

### BACKGROUND OF THE INVENTION

Hematopoietic stem cells (HSCs) are defined as cells with multi-lineage hematopoietic differentiation potential and sustained self-renewal activity. Operationally, HSCs are detected by their ability to regenerate long term multi-lineage hematopoiesis in myeloablated recipients. HSC numbers can be quantified by endpoints that measure this regenerative activity in genetically distinguishable, radio-protected hosts transplanted with limiting numbers of HSCs.¹ HSCs are also characterized by extensive heterogeneity. Variability in many HSC properties is dictated by changes in their state of activation and the consequent changes in these properties are thus reversible. For example, most of the HSCs present in normal adult mice are in a deeply quiescent (G₀) state²⁻⁴ and, in association with this status, they express CD38 but not CD34 or Mac1.^{5,6} These G₀ HSCs also actively exclude certain fluorescent dyes, such as rhodamine-123 (Rho)^{7,8} and Hoechst 33342 (Hst).⁹ The latter property underlies the detection of adult mouse HSCs as “side population” (SP) cells.¹⁰ However, when HSCs are activated, they rapidly down-regulate expression of CD38,^{6,11} increase expression of CD34¹² and Mac1^{13,14} and acquire a Rho-bright, non-SP phenotype.¹⁵ In association with these changes, some of the HSCs begin to differentiate and hence permanently lose their long term repopulating activity, but many do not, in spite of their transiently altered phenotype.¹⁶ Another property of HSCs that appears to vary reversibly is their ability to exit from the circulation into the bone marrow (BM) and re-initiate hematopoiesis. Quiescent adult mouse HSCs can execute this process at near unit efficiency in suitably myelosuppressed hosts, as shown by their ability to be detected at purities of ≧20% following intravenous injection.^{15;17;18} However, notable changes in HSC engrafting potential have also been found to accompany the progression of HSCs through the cell cycle both in vitro and in vivo.^{3;19-21} Specifically, HSC activity was not detectable in suspensions of adult or neonatal hematopoietic cells in S/G₂/M, even when substantial HSC activity could be found in the corresponding G₁ cells. The transient nature of the silencing of HSC homing activity during the progression of these cells through S/G₂/M is inferred from the fact that the populations studied did not contain G₀ HSCs, or were expanding their HSC content, although formal documentation of the re-acquisition of repopulating activity by incapacitated S/G₂/M HSCs was not documented. The molecular mechanisms that control the BM-homing activity of HSCs are not fully elucidated, although a number of cell surface ligand-receptor interactions with known involvement in cell adhesion and chemotaxis have been implicated. These include stromal cell-derived factor-1 (SDF-1; also known as CXCL12)/CXCR4, Steel factor (SF)/c-kit, CD44/hyaluronic acid (HA) or osteopontin (OPN), VLA-4/V-CAM1 and P2Y-like receptor and an unknown ligand.²²⁻²⁵ The expression and activity of some of these appear to be variably affected by cytokine exposure;^{26;27} however, their specific involvement in the engraftment defect of HSCs in S/G₂/M has remained unclear.

In the mouse embryo, pluripotent hematopoietic cells with long term repopulating ability first appear in the aorta-gonado-mesonephros (AGM) region on the 9th day of gestation.²⁸ These cells then migrate to the fetal liver (FL) and later to the BM with continuing expansion of their numbers until young adulthood is reached.²⁹ Most of the HSCs in the day 14.5 FL have phenotypic characteristics of activated adult HSCs (CD38−, Mac1+, CD34+, Rho+, non-SP)^{13;14}, as might be expected for an expanding HSC population. The proportion of HSCs in the day 14.5 FL that are proliferating has been previously estimated from phenotyping studies to be ˜35%,³ although a more direct measurement of this fraction has not been reported.

The mechanisms that regulate changes in stem cell turnover and engraftment properties during ontogeny have not yet been elucidated.

Normal HSCs of human²⁰ as well as mouse¹⁹ origin do not engraft in vivo when they transit the S/G₂/M phases of the cell cycle. This has little bearing on the therapeutic utility of hematopoietic transplants from normal adult bone marrow, mobilized blood or cord blood sources because the HSCs present in these sources are largely in G₀ or G₁. However, in the future, increasing use of HSCs stimulated to enter S/G₂/M ex vivo is envisioned. Such applications are being currently developed to expand the number of HSCs available for transplant where the initial number of HSCs available is insufficient and/or a gene correction manipulation is to be undertaken prior to transplantation of the cells. In such situations, strategies that would allow the S/G₂/M HSCs in these transplants to engraft would significantly enhance the utility of the transplants as the reconstituting activity would be increased.

HSC transplants are performed in patients requiring either transient and/or permanent rescue of their blood forming system. Cells for transplant may be obtained either from a histocompatible normal donor (allogeneic transplant) or the recipient may serve as his/her own donor (autologous transplant). The most common rationale for performing a HSC transplant is to ensure reconstitution of the blood-forming system in a patient with a malignancy requiring radiotherapy or chemotherapy at dosage levels that result in ablation of the blood-forming system. The second rationale is to replace a defective blood-forming system with normal cells, for example, immune deficiencies, aplastic anemia, autoimmune disorders, or certain inherited disorders. The normal cells may be derived from a normal donor or after genetic correction of the donor's own cells. In either scenario, the number of HSCs available may be limiting. Under these circumstances, a transplant may be precluded altogether, or may be attempted but with an increased risk of medical complications, or the possibility of graft failure. Thus, there is a need for strategies to increase the number of effective HSCs in a transplant. Successful strategies would increase significantly the number of patients who could benefit from a therapy that requires the transplantation of HSCs.

Current technology is available to expanding the populations of HSCs ex vivo using appropriate culture conditions and factors to stimulate their growth. Similarly gene therapy strategies require ex vivo activation and/or proliferation of the HSCs targets in order for these cells to incorporate the introduced corrective gene. However the efficiency of engraftment of proliferating cells or cells in S/G₂/M is reduced, and thus such manipulations cannot be relied upon for successful transplantation results.

Chemokines are small cytokines that control a wide variety of biological and pathological processes, including those involved in immunosurveillance, inflammation, viral infection, hematopoietic cell regulation and the metastasis of cancer cells. One possible approach to mimic or block the biological effects of chemokines is to interfere with the interaction of a chemokine with its cognate receptor. To date, the search for small molecule chemokine receptor antagonists has shown some preliminary successes. Drug discovery programs have identified antagonists for a number of chemokine receptors of medical interest: CCR5 and CXCR4 for HIV infection; CXCR4 alone for hematopoietic stem cell mobilization, CCR1 and CXCR3 for rheumatoid arthritis, multiple sclerosis and psoriasis; CCR2 for atherosclerosis, CCR3 for asthma and allergy; and CXCR2 for chronic obstructive pulmonary disease, rheumatoid arthritis or psoriasis.^{77, 78}

Generation of HSCs and primitive hematopoietic progenitors is regulated by cellular and humoral interactions in which chemokines play a crucial role. Among the chemokines, stromal cell-derived factor-1 (SDF-1, also known as CXCL12) and its receptor, CXCR4, have been reported to be key players in the homing of circulating HSCs into and the retention of HSCs within the bone marrow.⁷⁹ Disruption of the SDF-1\CXCR4 axis results in cell mobilization and may participate in leukaemia extramedullary infiltration. The SDF-1 chemokine and its receptor also have a number of other functions in the regulation of hematopoiesis, including regulation of their cell cycle status. The chemokines MCP-1 and MIP-1α have similar effects on slightly more mature types of primitive hematopoietic progenitors that include those identified as long-term culture initiating cells and primitive erythroid and granulopoietic colony-forming cells. Because of their pleiotropic effects on cell trafficking, survival and proliferation, modulation of the functions of these chemokines is a promising molecular target for the improvement of treatments that affect the functioning of the hematopoietic system.

SDF-1 has been shown to have direct effects on HSCs. SDF-1 has been shown to inhibit HSC entry into S phase both in vitro⁸⁰ and in vivo.³⁷ SDF-1 has also been observed to enhance platelet recovery in NOD/SCID mice transplanted with human cord blood cells and to mobilize primitive human progenitors into the blood.⁸¹

SDF-1 antagonists, such as AMD3100, have been developed for the mobilization of HSCs to enable the collection of cells from donors for transplantation.⁸² The SDF-1 antagonist AMD3100 combined with granulocyte-colony stimulating factor (G-CSF) for autologous hematopoietic progenitor cell mobilization is superior to G-CSF alone in clinical tests.⁷² SDF-1/CXCR4 antagonists have been assessed for clinical application in blocking HIV infection, preventing metastasis, treatment of arthritis and allergy, or preserving cardiac function after acute myocardial infarction.⁴³

A number of patent publications relate to chemokines, receptor antagonists, or agonists for SDF-1/CXCR4 and their use in treatment of conditions ranging from autoimmune disorders to cardiovascular disease. For examples of such patents and applications, see U.S. Patent Publication 2005/0164935; U.S. Patent Publication 2005/0059584; U.S. Patent Publication 2004/0197303; U.S. Patent Publication 2003/0148940; U.S. Patent Publication 2002/0165123; U.S. Patent Publication 2002/0156034; U.S. Patent Publication 2005/0080267; U.S. Pat. No. 6,613,742; U.S. Pat. No. 6,428,970; and U.S. Pat. No. 6,872,714, each of which is herein incorporated by reference. None of the patents or applications addresses the problem that stem cells in a proliferative phase experience reduced engraftment efficiency.

Glimm et al. (Blood 2000; 96 (13) p. 4185-4193) found that human hematopoietic stem cells stimulated to proliferate in vitro lose engraftment potential during their S/G₂/M transit. Thus, factors that act to induce proliferation of stem cells would be expected to have a detrimental effect on the engraftment efficiency.

Tudan et al., in U.S. Patent Publication 2001/0156034 teaches the use of a peptide antagonist to stimulate multiplication of hematopoietic cells. However, this does not address the problem of reduced engraftment, and renders a cell population in a proliferative phase that is less likely to successfully engraft than had the cells not undergone multiplication.

U.S. Patent Publication 2002/0094327 to Petersen describes a method of targeting a stem cell to a target tissue that involves increasing the concentration of SDF-1α within a tissue, or increasing the concentration of a SDF-1α agonist in the target tissue. This work suggests that SDF-1α presence or activity assists in engraftment of stem cells.

Cashman et al., (J. Exp. Med; 2002; 196 (9); 1141-1149) found that in vivo treatment of hematopoietic stem cells with SDF-1 has the potential for beneficial effects on transplantability of the cells removed and then injected into another recipient.

Li et al. (Stem Cells, Aug. 25, 2005-0082) describes the effect of SDF-1 peptide analog CTCE-0214, a SDF-1 agonist, on encouraging expansion of cord blood CD34⁺ cells. Cell cells expanded in the presence of CTCE-0214 showed some improvements in engraftment, consistent with what may be expected of an agonist, based on results obtained with SDF-1.

There is a need for enhanced engraftment efficiency of proliferating or S/G₂/M phase populations of stem cells, and in particular hematopoietic stem cells. Improvements are needed for stem cells that are provided by transplantation, or for naturally circulating HSCs. Such stem cell populations are currently incapable of engrafting efficiently in a subject.

### SUMMARY OF THE INVENTION

It is an object of the present invention to obviate or mitigate at least one disadvantage or difficulty encountered in previous attempts to transplant proliferating or S/G₂/M phase populations of stem cells.

In one aspect of the invention, there is provided a use of a SDF-1 antagonist for improving engraftment of stem cells in S/G₂/M phase in a recipient in need thereof. Thus, there is provided a use of a SDF-1 antagonist for preparation of a medicament for improving engraftment of stem cells in S/G₂/M phase in recipient in need thereof.

In a further aspect of the invention, there is provided a composition for transplantation of stem cells comprising S/G₂/M phase stem cells and a SDF-1 antagonist.

Another aspect of the invention provides a method of treating stem cells intended for transplantation, to improve engraftment, the method comprising the steps of: (a) inducing stem cells ex vivo to proliferate or enter S/G₂/M phase; and (b) providing a SDF-1 antagonist to proliferating or S/G₂/M phase stem cells.

Additionally, provided herein is a method of improving engraftment of S/G₂/M phase stem cells comprising the step of treating ex vivo expanded stem cells with a SDF-1 antagonist prior to delivery to a recipient. A method of improving engraftment upon transplant of S/G₂/M phase stem cells comprising the step of treating a transplant recipient with a SDF-1 antagonist prior to transplantation of ex vivo expanded stem cells is also provided.

In yet a further aspect of the invention, there is provided a method of transplanting S/G₂/M phase stem cells comprising the steps of: (a) obtaining stem cells; (b) inducing stem cells ex vivo to proliferate or enter S/G₂/M phase; (c) treating stem cells or recipient with a SDF-1 antagonist; and (d) providing stem cells to the recipient.

Additionally, an aspect of the invention provides the use of a SDF-1 antagonist for improving engraftment of a cell in S/G₂/M phase upon delivery to a recipient in need thereof, wherein engraftment of the cell is impacted by the SDF-1/CXCR4 pathway. Also provided is the use of a SDF-1 antagonist for preparation of a medicament for improving engraftment of a cell in S/G₂/M phase upon delivery to a recipient in need thereof, wherein engraftment of the cell is impacted by the SDF-1/CXCR4 pathway.

According to yet another aspect of the invention there is provided a method of improving engraftment upon transplant S/G₂/M phase cells for which engraftment is impacted by the SDF-1/CXCR4 pathway, the method comprising the step of treating a transplant recipient with a SDF-1 antagonist prior to transplantation of ex vivo expanded cells.

The beneficial effect of SDF-1 antagonists on transplant efficiency of stem cells was unexpected because previous reports suggest that SDF-1 itself or agonists of SDF-1 can enhance the engraftment of HSCs.

Advantageously, the method of the present invention improves engraftment efficiency by treatment of recipients of hematopoietic stem cell transplants with antagonists of SDF-1. Thus, improved outcomes of stem cell transplant can be realized for patients with cancer or other disorders such as immune deficiencies, aplastic anemia or autoimmune disorders where the availability of the appropriate donor material is limited.

Advantageously, chemokine antagonists of SDF-1 allow improved engraftment of naturally circulating or transplanted cells in a proliferative or S/G₂/M phase. Advantageously, embodiments of the invention will improve blood cell recovery, and increase the likelihood of survival of recipients of stem cell transplants. This will have impact in both clinical and research settings.

As a further advantage, improvements in engraftment, or the efficiency of engraftment, of transplanted proliferating or S/G₂/M phase hematopoietic stem cells by using chemokine SDF-1 antagonists will allow manipulation of stem cells prior to transplantation. Such manipulations may include genetic manipulation, or growth in the number of cells to be transplanted in the case where only a small number of cells is available. Previously, manipulation of cells in this way actually decreased the efficiency of engraftment for the manipulated population of cells. Embodiments of the invention improve the engraftment efficiency for proliferating or S/G₂/M phase cells, which opens new opportunities for successful transplantation after manipulation, or from small populations of cells. The number of stem cells available would not necessarily be a limiting factor in the decision to go forward with transplantation, and likelihood of medical complications and/or the possibility of graft failure would be reduced.

An advantage realized according to an embodiment of the invention is that an increased supply of stem cells becomes available for transplant by expanding the populations of stem cells ex vivo using appropriate culture conditions and factors to stimulate their division and growth. These cells are now a more appropriate population for transplantation due to the effect of exposure to the SDF-1 antagonist.

A further advantage realized according to an embodiment of the invention is that gene therapy strategies, requiring ex vivo activation and/or proliferation of the target stem cells to incorporate the introduced corrective gene, can result in a higher engraftment efficiency. Thus, one road-block to successful transplantation after genetic manipulation is addressed.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1A is a schematic illustration of a method for improvement of the engraftment efficiency of stem cells by treatment of the recipient.

FIG. 1B is a schematic illustration of a method for improvement of the engraftment efficiency of stem cells by treatment of the stem cells prior to delivery to a recipient.

FIG. 2 illustrates competitive repopulating units (CRU) of cells derived from different mouse embryonic tissue after 16 hour in vivo or in vitro treatments.

FIG. 3 shows fluorescence activated cell sorter (FACS) profiles of the distribution of different lin⁻ populations in G₀, G₁ and S/G₂/M.

FIG. 4 depicts cycling activity of CRUs, showing the number of CRUs per 10⁵ initial total viable cells, demonstrating down-regulation between 3 and 4 weeks of age.

FIG. 5 illustrates that Hst/Py-sorted HSCs display an absolute but transient S/G₂/M engraftment defect. In part A, E14.5 Ter119⁻ FL (fetal liver) cells in G₁/S/G₂/M were fractionated into their component G₁ and S/G₂/M subsets. In part B, CRUs per 10⁵ initial Ter119⁻ FL cells for G₁ and S/G₂/M fractions are shown before and after 6 hours in culture.

FIG. 6 shows the impact of in vivo (part A) and in vitro (part B) treatments with SDF-1G2 on engraftment defect.

FIG. 7 shows donor-derived repopulation of SDF-1G2-treated mice for G₁ cells in PBS-treated mice (part A); S/G₂/M cells in SDF-1G2-treated mice (part B); and in S/G₂/M cells in PBS-treated mice (part C).

FIG. 8 shows Gene expression analysis of the G₁ and S/G₂/M subsets of highly purified lin⁻Sca1⁺CD43⁺Mac1⁺ HSCs from fetal liver (FL) and 3-week bone marrow (BM).

### DETAILED DESCRIPTION

In one aspect of the present invention, it provides a method for improving the engraftment of proliferating stem cells. This method would provide improved blood cell recovery, improved survival and related benefits in recipients of such cells in both clinical and research settings.

The term “SDF-1 antagonist” as used herein encompasses an antagonist that targets stromal cell-derived factor-1 (SDF-1) and/or its receptor, CXCR4, or an antagonist that targets an element that regulates the production, secretion or function of SDF-1 in vivo. Such an antagonist may be an agent that interacts with CXCR4, or SDF-1 in such a way that results in antagonism of SDF-1. Additionally, an “SDF-1 antagonist” can encompass the antagonistic effect resulting from the introduction of DNA encoding antisense RNA to CXCR4, RNA interference (RNAi), other nucleic acids or TAT fusion proteins into stem cells selected for transplantation, or for treatment of or delivery to a stem cell recipient. Specific exemplary antagonists are discussed in detail below.

As used herein, the term “stem cells” is used to refer to hematopoietic stem cells or other types of stem cells or progenitor cells capable of repopulating bone marrow, or utilizing the SDF-1/CXCR4 pathway to enter tissues. Thus, the term “stem cells” may additionally encompass neuronal stem cells, breast stem cells, gut stem cells, or skin-associated stem cells. Further specific examples are described below.

The terms “engraftment” or “engraftment efficiency” are used interchangeably herein to mean any effects resulting in the ability of stem cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency can be evaluated or quantified using any clinically acceptable parameter, for example, by assessment of competitive repopulating units (CRU); incorporation or expression of a marker in tissue(s) into which stem cells have homed, colonized, or become engrafted; or by evaluation of the progress of a subject through disease progression, survival of stem cells, or survival of a recipient.

The phrase “ex vivo expanded stem cells” as used herein relates to a population of stem cells that have been induced to proliferate (and thus expand in numbers) while not contained within a subject, either donor or recipient. Any method of inducing proliferation that would be acceptable to a person skilled in the art could be employed to expand the stem cells. Once expanded, the resulting stem cells may be in a proliferative state, or may be in any of S/G₂/M phases. Some cells within the expanded population may not have undergone proliferation, and/or may be in a G₀ or G₁ phase. Individual cells within the population so expanded may be at different phases relative to one another.

Stem cells which can be used according to the invention may be either naturally circulating cells or cells intended for transplant. Cells for transplant are considered those which are removed from a donor, or are obtained from culture, and which are provided to a recipient. Stem cells to be transplanted may be obtained either from a histocompatible normal donor (allogeneic transplant) or the recipient may serve as his/her own donor (autologous transplant).

Stem cells circulating in peripheral blood can be treated according to the invention and may be enticed to home to tissues rather than circulate.

The hematopoietic stem cells used may be derived from any one or more of the following sources: fetal tissues, cord blood, bone marrow, peripheral blood, mobilized peripheral blood, a stem cell line, or may be derived ex vivo from other cells, such as embryonic stem cells or adult pluripotent cells. The cells from the above listed sources may be expanded ex vivo using any method acceptable to those skilled in the art prior to use in the transplantation procedure. For example, cells may be sorted, fractionated, treated to remove malignant cells, or otherwise manipulated to treat the patient using any procedure acceptable to those skilled in the art of preparing cells for transplantation. If the cells used are derived from an immortalized stem cell line, further advantages would be realized in the ease of obtaining and preparation of cells in adequate quantities.

Cells which may be treated or used according to the invention include cells in the hematopoietic lineage, including pluripotent stem cells, bone marrow stem cells, progenitor cells, lymphoid stem or progenitor cells, myeloid stem cells, CFU-GEMM cells (colony-forming-unit granulocyte, erythroid, macrophage, megakaryocye), B stem cells, T stem cells, DC stem cells, pre-B cells, prothymocytes, BFU-E cells (burst-forming unit—erythroid), BFU-MK cells (burst-forming unit—megakaryocytes), CFU-GM cells (colony-forming unit—granulocyte-macrophage), CFU-bas cells (colony-forming unit—basophil), CFU-Mast cells (colony forming unit—mast cell), CFU-G cells (colony forming unit granulocyte), CFU-M/DC cells (colony forming unit monocyte/dendritic cell), CFU-Eo cells (colony forming unit eosinophil), CFU-E cells (colony forming unit erythroid), CFU-MK cells (colony forming unit megakaryocyte), myeloblasts, monoblasts, B-lymphoblasts, T-lymphoblasts, proerythroblasts, neutrophillic myelocytes, promonocytes, or other hematopoietic cells that differentiate to give rise to mature cells.

Cells that are proliferating or that are in S/G₂/M phase may be naturally dividing or may be induced or stimulated to divide, for example, through treatment ex vivo prior to use in a transplantation procedure. The enhanced engraftment efficiency of stem cells is particularly beneficial for proliferating cells or cells in S/G₂/M phase, which have been induced to divide ex vivo, but the effect is not limited to such cells. Cells which may be used with the invention, and which may realize an improved engraftment include those cell types where engraftment is impacted by the SDF-1/CXCR4 pathway. By impacted, it is meant that engraftment can regulated or controlled according to the SDF-1/CXCR4 pathway.

In addition to those stem cells in S/G₂/M or a proliferative state, the invention may be used with any stem cell that is in a non-engrafting state, or is resistant to engrafting. Cells in a G₁ phase that are resisting engraftment may benefit from embodiments of the methods according to the invention. Cultured cells, which may traditionally have reduced engraftability may have modified engraftment success according to the invention.

The cells intended for enhanced engraftment may be cells which have been modified or corrected by gene therapy. In such an instance, the cells may have previously been induced to a proliferative state so as to incorporate a gene of interest. In this instance, cells may be allogenic or autologous.

The SDF-1 antagonist may be a protein, an antibody, a peptide, RNAi, an antisense nucleotide, a small or low molecular weight molecule or drug. An exemplary SDF-1 antagonist for use with the invention may be a chemokine. A further exemplary SDF-1 antagonist may be a protein.

An exemplary chemokine antagonist of SDF-1 is AMD3100, and related compounds or analogs, for example as described in U.S. Pat. No. 6,667,320 entitled “Chemokine receptor binding heterocyclic compounds” to AnorMED Inc., (Vancouver, Canada). AMD3100 is a member of a bicyclam class of compounds. AMD3100 can be represented as the octahydrochloride dihydrate of 1,1′-[1,4-phenylene-bis-(methylene)]-bis-1,4,8,11-tetra-azacyclotetradecane, and has a molecular weight of 830. Originally, AMD3100 (also known as JM3100) was considered to be a potential anti-HIV therapeutic.⁴¹ Other compounds within the bicyclam class which may have similar effects to AMD3100 on SDF-1 antagonism would also fall within the general term “SDF-1 antagonists”.

An exemplary chemokine antagonist having a similar sequence to SDF-1, referred to herein as SDF-1G2, may be used according to embodiments of the invention. SDF-1 is identical to SDF-1 except that the proline at position 2 has been converted to glycine⁴². Another form of notation for SDF-1G2 is SDF-P2G, acknowledging the substitution of glycine for proline. Chemokine antagonist SDF-1G2 is a protein having a sequence according to the sequence shown below, a 67-amino acid chain in which glycine is present at position 2: KGVSL SYRCP CRFFE SHVAR ANVKH LKILN TPNCA LQIVA RLKNN NRQVC IDPKL KWIQE YLEKA LN (SEQ ID NO:1). Analogs or active portions of this sequence which may be used to achieve the same antagonistic effect as SDF-1G2 are also considered to fall within the term “SDF-1 antagonists”. For example, proteins having amino acid sequences bearing 85% or greater identity to SDF-1G2, for example, having 90% or greater or 95% or greater identity to SDF-1G2 and having SDF-1 antagonistic effects would fall within the meaning of the general term “SDF-1 antagonists”. As an example of other peptide antagonists, TC14012 may be used (see, for example Juarez et al., Leukemia 2003; 17:1294-300). Any analog of SDF-1 that can behave in an antagonistic manner to the SDF-1/CXCR4 pathway may be of use according to the invention.

The present invention may be used to improve outcomes of stem cell transplant for patients with cancer or other disorders such as immune deficiencies, aplastic anemia or autoimmune disorders where the availability of the appropriate donor material is limited.

The inventive method may be useful in treatment of transplant-associated disorders caused by poor engraftment of hematopoietic stem cells. Such disorders include but are not limited to abnormal migration of hematopoietic cells, hematopoietic stemcytopenia after bone marrow transplantation, leukocytopenia, neutropenia, thromocytopenia, leukopenia, or lymphopenia after chemotherapy. Further, the invention may be useful for the treatment of leukemia, for treatment or prevention of extramedullary infiltration by leukemic cells, or for treatment of the metastasis of malignant cells. Additionally, SDF-1 antagonists may be used to enhance engraftment of a variety of types of stem cells, other than hematopoietic stem cells during organ or tissue transplantation.

In addition to benefits realized through treatment or prevention disease, SDF-1 antagonists may be used to prepare a stem cell population suitable for use in drug screening projects designed to identify inhibitors of leukemic or other types of metastatic malignant cells. Such cells would have an enhanced engraftment phenotype that is associated with metastatic cells, and thus could be used to identify effective inhibitors.

An exemplary method according to the invention involves treatment of the recipient, or prospective recipient of stem cells with a SDF-1 antagonist prior to infusion of stem cells for transplant. Such treatment may be before, during, or after transplant, or a combination of these may be employed. Mode of delivery of the antagonist may be according to any pharmaceutically acceptable regime, and dosage form.

When delivered to the recipient, the SDF-1 antagonist may be provided in any pharmaceutically acceptable dosage form at effective levels that fall within medically acceptable ranges. For example, from 0.001 to 10 mg/kg of an antagonist peptide may be used, and a further exemplary range would be from 0.01 to 1 mg/kg. For a small molecule antagonist, such as AMD3100, or analogs thereof, an exemplary dosage may be from 0.001 to 100 mg/kg, and a further exemplary range may be from 0.01 to 10 mg/kg. The delivery method may be by infusion, injection, via a targeted delivery vehicle, or through any method acceptable to those skilled in the art.

A further exemplary method according to the invention involves treatment of stem cells ex vivo, prior to transplantation, in such a way that will be antagonistic to SDF-1. In a specific example, cells may be exposed to a chemokine antagonist of SDF-1 prior to transplantation. This exposure of ex vivo expanded stem cells may be provided according to any appropriate therapeutic regime.

Further, a combination approach may be used in which cells are treated with a SDF-1 antagonist ex vivo prior to delivery to a recipient, and a recipient may be treated either pre- or post delivery of stem cells with a SDF-1 antagonist. In such a combination approach, it is not necessary to employ the same SDF-1 antagonist for each stage. For example, the cells may be treated ex vivo with one type of antagonist, whereas the recipient may receive a different type of antagonist.

Specific details are set forth herein in order to provide a more thorough understanding of the invention. However, the invention may be practiced without these particulars, or by incorporating equivalents without departing from the spirit and scope of the invention. In some instances, well known elements have not been shown or described in detail, as these would be understood by a person skilled in the art. The examples and drawings are to be regarded in an illustrative, rather than a restrictive sense. All published documents referred to herein are incorporated by reference.

### EXAMPLE 1

### Enhancement of Engraftment Efficiency of Transplanted Stem Cells by Treatment of Recipient

An exemplary method is provided herein to enhance engraftment efficiency of stem cells transplanted into a recipient after the stem cells have been induced to proliferate or enter S/G₂/M phase ex vivo.

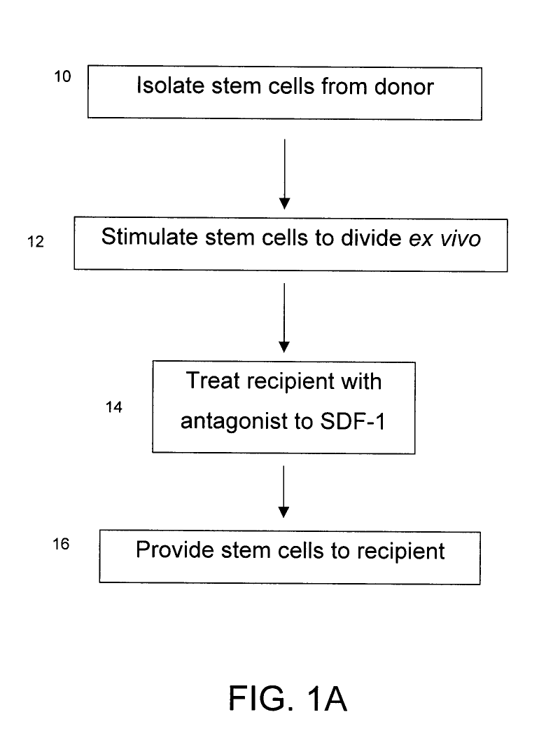

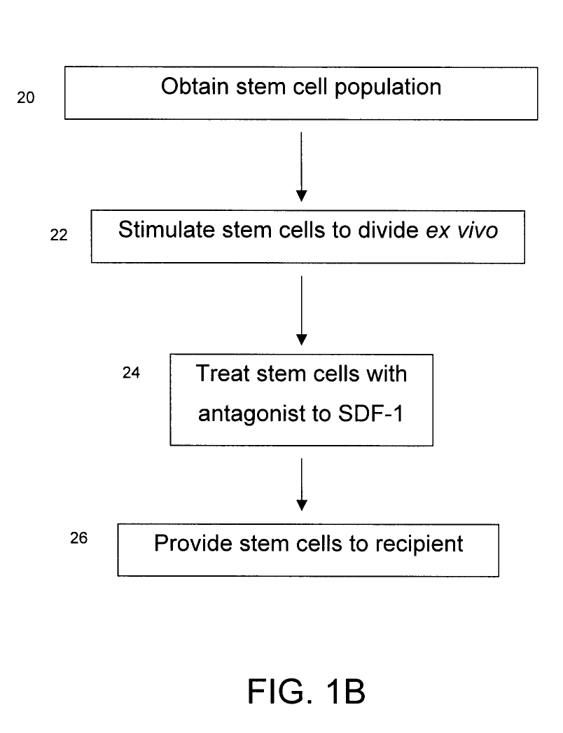

FIG. 1A illustrates the method employed. Stem cells are obtained by isolation of cells from the donor (10), for example by collection of mobilized peripheral blood or cord blood. Following isolation, the stem cells are cultured in the presence of suitable media and growth factors to stimulate the population of stem cells to divide ex vivo (12). Prior to delivery of the stimulated stem cells, the recipient is treated (14) with a SDF-1 antagonist using a treatment dosing schedule in an amount adequate to enhance engraftment of the stem cells. The stem cells are then provided to the recipient (16) by infusion or injection. Typically, the recipient is a subject that has been treated to purge their bone marrow of preexisting stem cells or to eradicate malignant cells.

By treatment of the recipient in advance of the provision of stem cells, the environment to which the stem cells home and colonize is rendered conducive to engraftment. Treatment of the recipient with a SDF-1 antagonist during and after infusion of stem cells is also an optional component of the treatment, in order to realize beneficial effects.

### EXAMPLE 2

### Enhancement of Engraftment Efficiency of Transplanted Stem Cells by Treatment of Stem Cells Ex Vivo

An exemplary method is provided herein to enhance engraftment efficiency of stem cells transplanted into a recipient after the stem cells have been induced to proliferate or enter S/G₂/M phase ex vivo.

FIG. 1B illustrates the method employed. A population of stem cells is obtained (20) for example by isolating cells derived from a donor. This may be done by collection of mobilized peripheral blood or cord blood. The stem cells obtained are cultured in the presence of suitable media and growth factors to stimulate the population of stem cells to divide ex vivo (22). The stimulated stem cells are treated (24) with a SDF-1 antagonist at a concentration adequate to enhance engraftment of the stem cells. The stem cells are then provided to the recipient by infusion or injection (26). Typically, the recipient is a subject that has been treated to purge their bone marrow of preexisting stem cells or to eradicate malignant cells.

By treatment of the stem cells ex vivo with a SDF-1 antagonist before providing the stem cells to the recipient, the stem cells will have derived benefit from exposure to SDF-1, and the population delivered to the recipient has already been rendered conducive to engraftment. Optionally, treatment of the recipient with a SDF-1 antagonist before, during or after infusion of antagonist-treated stem cells may be used to further enhance engraftment efficiency.

### EXAMPLE 3

### Hematopoietic Stem Cells Proliferate Until After Birth and Show a Reversible Phase-Specific Engraftment Defect

In order to illustrate the effect of SDF-1 antagonists on improving engraftment, experiments are described herein which assess HSC proliferative status in mice at different stages of development. This experiment shows that the entire HSC population remains in cycle until the 3rd week after birth regardless of the tissue in which the HSCs are located. Then within one week, the majority of the HSCs switch abruptly from an actively dividing to a quiescent state. Until this switch occurs, those HSCs that are in S/G₂/M show the same engrafting defect previously demonstrated for adult HSCs that have been stimulated to divide. Interestingly, prior to the establishment of a quiescent HSC population, the HSCs in S/G₂/M were found to express higher levels of SDF-1 than those in G₁ and their defective engrafting activity could be completely reversed, either by holding them ex vivo for a few hours until they re-entered G₁, or by pretreating the host with an antagonist of stromal cell-derived factor-1 (SDF-1).

By way of background, the regulation of hematopoietic stem cell (HSC) proliferation and engraftment of the bone marrow is an important but poorly understood process, particularly in terms of changes that occur during ontogeny. In mice, all HSCs are cycling until 3 weeks after birth. Then, within one week, most become quiescent. Prior to 4 weeks of age, the proliferating HSCs with long term multi-lineage repopulating activity display an engraftment defect when transiting S/G₂/M. During these cell cycle phases their expression of stromal cell-derived factor −1 (SDF-1) transiently increases. The defective engrafting activity of HSCs in S/G₂/M is reversed if, prior to injection, the cells are allowed to progress into G₁, or if the hosts (but not the cells) are pretreated with an antagonist of SDF-1. Interestingly, the enhancing effect of pretreating the hosts with a SDF-1 antagonist is exclusive to transplants of long term multi-lineage repopulating HSCs in S/G₂/M. These results demonstrate a new HSC regulatory checkpoint during development. They also indicate a previously unrecognized ability of HSCs to express SDF-1 in a fashion that changes with cell cycle progression and is associated with a defective engraftment that can be overcome by in vivo administration of a SDF-1 antagonist.

The following non-standard abbreviations are used herein: α4int—α4-integrin; AGM—aorta-gonado-mesonephros; CRU—competitive repopulating unit; FL—fetal liver; FACS—fluorescence activated cell sorting; 5-FU—5-fluorouracil; Gapdh—glyceraldehyde-3-phosphate dehydrogenase; HA—hyaluronic acid; HF/2—Hanks balanced salt solution containing 2% FCS; ³H-Tdr—³H-thymidine; Hst—Hoechst 33342; OPN—osteopontin; p.c.—post coitus; Py—pyronin Y; Rho—rhodamine-123; SDF-1—stromal cell-derived factor-1; SEM—standard error of the mean; SF—Steel factor; and SP—side population.

Methods

Mice. Ly5-congenic strains of C57BI/6 mice were used as donors and recipients.

All recipients were also homozygous for the W41 allele. Mice were bred and maintained in microisolators with sterile food, water and bedding at the BC Cancer Research Centre according to protocols approved by the University of British Columbia Animal Care Committee.

Cells. Single cell suspensions were prepared in Hank's balanced salt solution containing 2% fetal calf serum (FCS) (HF/2, StemCell Technologies). Enriched populations of HSCs were obtained by immunomagnetic removal of Ter119⁺ or lin⁺ cells from FL and BM cell suspensions, respectively (using EasySep™, StemCell Technologies). Antibodies used for isolation of lin⁻ cells between 4 and to 10 weeks of age were anti-B220, Ter119, anti-Gr1, anti-Ly1 and anti-Mac1 (StemCell Technologies). To isolate lin⁻ cells from 3 week-old mice, the Mac1 antibody was omitted because Mac1 was known to be expressed on fetal and cycling HSCs.^{13;14;73}

Tritiated ³H-Tdr suicide assay. Cells were suspended at 10⁶/ml in Iscove's medium containing 5×10⁻⁵ mol/l 2-mercaptoethanol, a serum substitute (BIT™, StemCell Technologies) and 50 ng/ml murine SF (StemCell Technologies). Equal volumes were then incubated at 37° C., in 5% CO₂ in air for 16 hours in 35 mm petri dishes in the presence or absence of 20 μCi/ml of ³H-Tdr (25 Ci/mmol; Amersham). The cells were then harvested, washed twice with Iscove's medium containing 2% FCS and limiting dilution CRU assays performed.

FACS isolation and analysis of cells in different cell cycle phases. Cells were suspended in HF/2 containing 1 μg/ml Hst (Molecular Probes/Invitrogen) either 10 μmol/l fumitremorgin C (a gift from Dr. Susan Bates, NIH, Bethesda, Md.) or 50 μmol/l reserpine (Sigma Chemicals) and then incubated at 37° C. for 45 minutes. PyroninY (Py; Sigma) was added at 1 μg/ml and the cells incubated for another 45 minutes at 37° C. Cells were washed twice in HF/2 with 1 μg/ml PI (Sigma) in the second wash and were finally resuspended in HF/2 with PI and kept on ice in the dark until being sorted (PI-cells only) on a 3 laser FACS Vantage (Becton Dickinson). For analysis of DNA content, cells were either re-stained with Hst only using the same protocol, or with PI at least 1 hour after storage at 4° C. of cells that had been washed twice in ice-cold PBS with 0.1% glucose and fixed in 1 ml of ice-cold 70% ethanol. To stain the cells with PI, cells were washed twice with 2% PBS and resuspended in PBS with 0.1% glucose, 5 μg/ml PI and 200 μg/mL RNAse A. Cells were then incubated for at least 1 hour at 4° C. and then analyzed directly on a FACSCalibur™ (Becton Dickinson). To stain sorted cells for Ki67, the cells were washed and resuspended in 50 μl of ice-cold 80% ethanol and then incubated at −20° C. for at least 2 hours. The fixed cells were washed twice in 300 μl of PBS with 1% FCS and 0.09% NaN₃ (pH=7.2). Fluorescein isothiocyanate (FITC)-conjugated anti-human Ki67 antibody (Becton Dickinson) was then added and the cells incubated for 30 minutes at room temperature in the dark. Cells were then analyzed by FACS, using cells stained with a FITC-conjugated mouse IgG1 (Becton Dickinson) as a control.

In vitro treatment of S/G₂/M HSCs. Sorted cells were incubated at 37° C. in 5% CO₂ in air in the wells of a round-bottom 96-well plate in serum-free media (as for the ³H-Tdr suicide assays) with one of the following 6 additions: 100 ng/ml SDF-1 (a gift from 1. Clark-Lewis, Biomedical Research Centre, University of British Columbia, Vancouver, BC, Canada) or 300 ng/ml SDF-1G2 (originally also obtained as a gift from I. Clark-Lewis, now available at cost as a requested peptide from the Biomedical Research Centre, University of British Columbia, Vancouver, BC, Canada) but no SF, or 50 ng/ml SF alone, or 50 ng/ml SF plus 100 ng/ml SDF-1 or 300 ng/ml SDF-1 or 300 ng/ml SDF-1G2. Cells were then harvested from the wells and equal aliquots injected into recipient mice such that each mouse received the equivalent of either 4×10³ starting G₁ cells or 1.2×10⁴ starting S/G₂/M cells.

CRU assay. Recipient (W41/W41) mice were sublethally irradiated with either 400 cGy of 137Cs γ-rays or 360 cGy of 250 kVp X-rays and then injected intravenously with the test cells except when injected intrafemorally as indicated. Intrafemoral injections were performed as described.⁷⁴ CRUs were identified by their ability to have generated ≧1% donor Ly5-type blood cells including Ly1+ (T-cell), B220+ (B-cell) and Gr1/Mac1+ (granulocyte/monocyte) subsets that could be detected ≧16 weeks after transplantation.⁷⁵ CRU frequencies were calculated using the L-calc program (StemCell Technologies) from the proportions of mice given various doses of test cells that were negative for this endpoint. Recipients treated with SDF-1G2 were injected intravenously with 10 μg per mouse of SDF-1G2 dissolved in PBS 2 hours after being irradiated and were then transplanted another 2 hours later. This schedule was used in an attempt to minimize direct interaction of the injected HSCs with SDF-1G2 in the circulation (based on the likely rapid clearance of SDF-1G2) and to maximize any potential effect on the host by keeping the interval between injecting the SDF-1G2 and the transplant as short as possible. Controls were injected with PBS instead of the SDF-1G2.

Real-time PCR. Cells were sorted into 1 ml HF/10 and RNA was isolated using the PicoPure™ RNA Isolation Kit (Arcturus Biosciences Inc.) as recommended by the supplier including a 15 minute DNAse1 treatment (Qiagen) on the column at room temperature. RNA was eluted into an 11 μl volume and stored at −80° C. A cDNA preparation was then generated using the SuperScript™ III First-Strand Synthesis System for RT-PCR (18080093, Invitrogen) again as recommended by the manufacturer, with the reaction scaled up to use 25 μl. Quantitative real-time PCR was performed using the following primer pairs (5′ to 3′): α4int (NM_{—}010576.2) forward primer AGGACACACCAGGCATTCAT (SEQ ID NO: 2), reverse primer CCTCAGTGTTTCGTTTGGTG (SEQ ID NO: 3); CD44 (NM_{—}009851.1) forward primer CTTTATCCGGAGCACCTTGGCCACC (SEQ ID NO: 4), reverse primer GTCACAGTGCGGGAACTCC (SEQ ID NO: 5); c-Kit (NM_{—}021099.2) forward primer ACAAGAGGAGATCCGCAAGA (SEQ ID NO: 6), reverse primer GAAGCTCAGCAAATCATCCAG (SEQ ID NO: 7); c-mpl (NM_{—}010823.1) forward primer AGTGGCAGCACCAGTCATCT (SEQ ID NO: 8), reverse primer GAGATGGCTCCAGCACCTT (SEQ ID NO: 9); CXCR4 (NM_{—}009911.2) forward primer CGGAGTCAGAATCCTCCAGT (SEQ ID NO: 10), reverse primer CTGGTCAGTCTCTTATATCTGGAAAA (SEQ ID NO: 11); Gapdh (NM_{—}008084) forward primer AACTTTGGCATTGTGGAAGG (SEQ ID NO: 12), reverse primer ATGCAGGGATGATGTTCTGG (SEQ ID NO: 13); SDF-1 (NM_{—}001012477) forward primer GAGCCAACGTCAAGCATCTG (SEQ ID NO: 14), reverse primer CGGGTCAATGCACACTTGTC (SEQ ID NO: 15); VCAM-1 (NM_{—}011693.2) forward primer TGATTGGGAGAGACAAGCA (SEQ ID NO: 16), reverse primer AACAACCGAATCCCCAACTT (SEQ ID NO: 17).

The relative expression changes were determined with the 2^{-ΔCT} method,⁷⁶ and the housekeeping glyceraldehyde-3-phosphate dehydrogenase (Gapdh) gene transcript was used to normalize the results.

Statistical analyses. Comparisons were made using the Wald test.

Results

All HSCs in the day 14.5 FL are rapidly proliferating. To measure the proportion of HSCs that are in cycle in the day 14.5 FL, we used 3 complementary strategies. In the first, we injected pregnant mice on day 13.5 p.c. with 100 mg/kg of 5-fluorouracil (5-FU) and then removed the fetuses 16 hours later, prepared cell suspensions from the FLs and measured the number of HSCs present using a limiting dilution transplantation assay for long term (16-week) competitive repopulating units (CRUs).¹

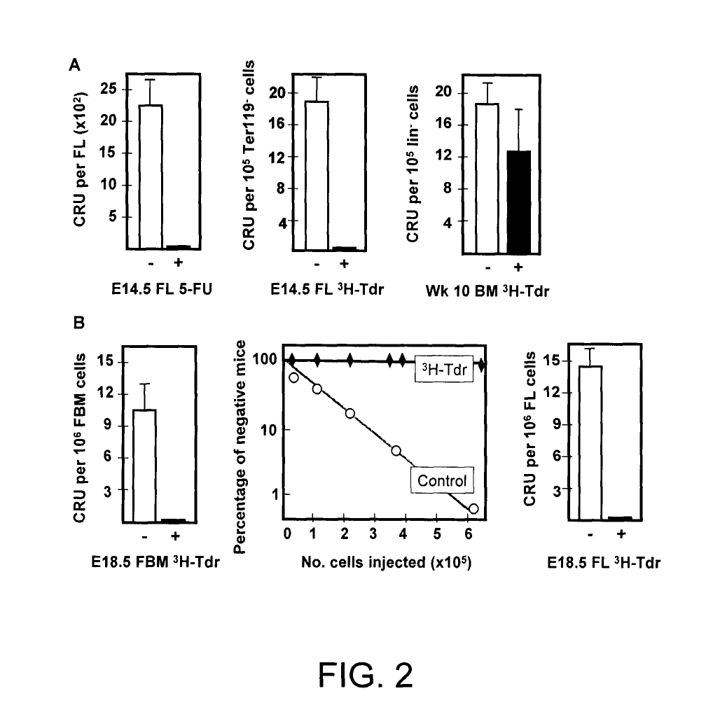

FIG. 2 illustrates that all fetal HSCs are sensitive to cell cycle-specific drugs. Cells from different mouse embryonic tissues were analyzed for CRU content either 16 hours after injection of the pregnant mother with 100 mg/kg 5-FU (or PBS), or after in vitro incubation of the cells for 16 hours with high-specific activity ³H-Tdr (or not). In Part A, the left panel shows the effects of the 5-FU injection on day 14.5 FL CRUs, data pooled from 3 independent experiments). The middle panel shows the effects of ³H-Tdr on day 14.5 FL CRUs and the right panel shows the (lack of) effect of ³H-Tdr on CRUs from adult (10 week-old) mice assessed in parallel (data pooled from 6 independent experiments). In Part B, the left and right panels show the effects of ³H-Tdr on day 18.5 fetal BM and FL CRUs (data pooled from 4 and 4 independent experiments, respectively). The middle panel shows the complete data set from the limiting dilution analysis of the day 18.5 fetal BM cells.

In these experiments, we detected very few CRUs in the FLs of the 5-FU-treated embryos (FIG. 2, Part A, left panel, 3 experiments). A comparison of the yields of CRUs from the 5-FU-treated FLs with the control FLs from pregnant mice injected on day 13.5 with phosphate buffered saline (PBS) indicated that the 5-FU treatment had reduced the expected CRU population in vivo by more than 1000-fold.

We then assessed the cycling status of HSCs in the day 14.5 FL by measuring the proportion of CRUs that survived a 16-hour exposure to high-specific activity ³H-thymidine (³H-Tdr).³⁰ Sixteen hours was anticipated to be sufficient to allow all cycling HSCs to enter S-phase, as confirmed later (see below), with minimal exit of any quiescent cells from G₀,³¹ as demonstrated for adult BM HSCs, most of which are in G₀ (FIG. 2, Part A, left panel). For these experiments, the Ter119⁺ (erythroid) cells were first removed from the FL cells to give a 10-fold enrichment in HSC content and the cells were then incubated in a serum-free medium supplemented with 50 ng/ml SF only. This growth factor condition was chosen based on other data demonstrating that freshly isolated day 14.5 FL CRUs are maintained at input numbers for 16 hours under these conditions. The results of the ³H-Tdr suicide experiments showed that this treatment reduced the number of CRUs in the suspensions of day 14.5 FL cells more than 100-fold (P<0.001, FIG. 2, Part A, middle panel), whereas the same treatment had no significant effect on the recovery of CRUs in similarly treated lineage-marker-negative (lin⁻) BM cells from young adult (10 week-old) mice by comparison to either control cells (incubated without ³H-Tdr, FIG. 1A, right panel, P=0.17) or to the starting values (data not shown).

The distribution of CRUs between the G₀ and G₁/S/G₂/M fractions of day 14.5 Ter119⁻ FL cells was then assessed. These subsets were isolated by fluorescence activated cell sorting (FACS) on the basis of their staining with Hst and Pyronin Y (Py).³²

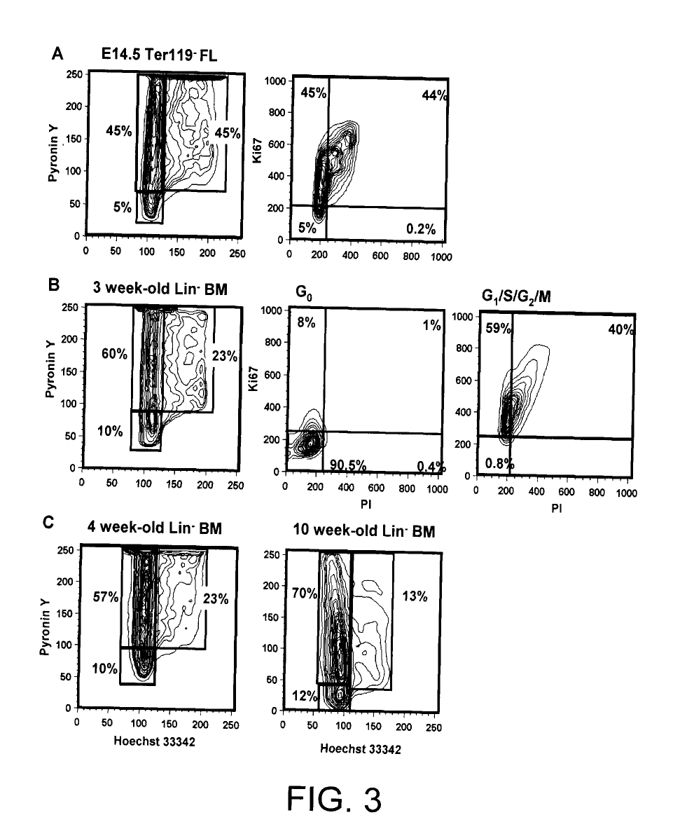

FIG. 3 shows FACS profiles of the distribution of different lin⁻ populations in G₀, G₁ and S/G₂/M. In Part A, the left panel shows a representative FACS contour plot for day 14.5 Ter119⁻ FL cells after staining with Hst and Py. The right panel shows the profile for the same cells after staining for Ki67. In Part B, the left panel shows a representative FACS contour plot for lin⁻ BM cells from 3 week-old mice after staining the cells with Hst and Py. The middle panel shows the profile for the sorted G₀ cells after staining for Ki67 (>90% of the G₀ cells showed no Ki67 expression). The right panel shows the profile for the sorted G₁/S/G₂/M cells after staining for Ki67 (>99% of the G₁/S/G₂/M cells expressed Ki67). In Part C, representative FACS contour plots are shown for lin⁻ BM cells from 4 week-old and 10 week-old mice after staining the cells with Hst.

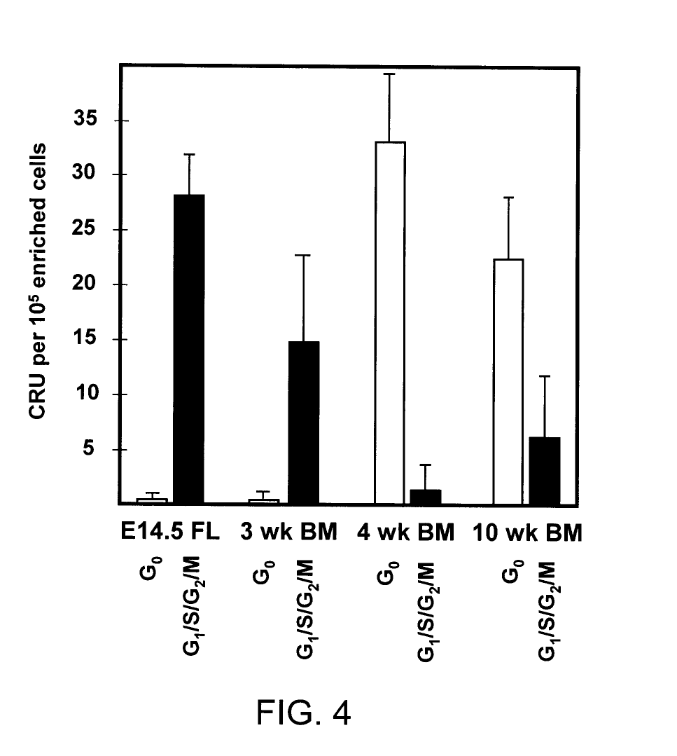

FIG. 4 shows that the cycling activity of CRUs is down-regulated between 3 and 4 weeks of age. Results shown are the number of CRUs per 10⁵ initial total viable cells. For each tissue source, the difference in the yields of CRUs in the 2 subsets compared was significantly different (P<0.001). For FL, these were depleted of Ter119⁺ cells; for the 3 and 4 week-old BM cells, all lin+ cells except Mac1+ cells had been removed and, for the 10 week-old BM cells, all lin+ cells including Mac1+ cells were removed. Values shown are the mean±SEM from data pooled from at least 3 experiments per tissue.

FIG. 4 shows the combined results of in vivo assays of the sorted cells from 4 independent experiments. These data indicate that all of the transplantable CRU activity was confined to the G₁/S/G₂/M fraction. Based on the total number of G₀ cells assayed, the proportion of quiescent HSCs could be estimated to be less than 0.02%.

HSCs undergo a complete and abrupt change in cycling activity between 3 and 4 weeks after birth. Since HSCs are known to be present in the BM of fetal mice at later times of gestation, it was of interest to investigate whether HSCs first become quiescent in the fetus at that site. To address this question, we used the 16-hour ³H-Tdr suicide assay to determine the cycling status of the CRUs present in the BM of mice at day 18.5 p.c. For comparison, we also evaluated the cycling status of CRUs in the day 18.5 FL.

Table 1 shows that the frequency of CRUs in these 2 tissues was 1 per 10⁵ and 1 per 7×10⁴ total nucleated cells, i.e., ˜5 and 3.5-fold lower than in adult BM (1 per 2×10⁴ total nucleated cells³³), and 6 and 4-fold lower than in the day 14.5 FL (1 per 1.7×10⁴ total nucleated cells³³). After overnight exposure to high-specific activity ³H-Tdr, no CRUs could be detected in the suspensions of either the day 18.5 fetal BM cells or the day 18.5 FL cells, in contrast to the control cells incubated in the same medium without ³H-Tdr (FIG. 2, Part B). Thus all HSCs in the fetus, irrespective of their location, appear to be rapidly proliferating.

TABLE 1
Limiting dilution data for CRU frequency determinations
for day 18.5 FL and BM cells (data pooled from 5 experiments).

| FL | | BM | |
|---|---|---|---|
| | | | Negative mice/ |
| Cells injected/ | Negative mice/ | Cells injected/ | total mice |
| mouse (×10³) | total mice injected | mouse (×10²) | injected |
| 2800 | 1/9 | 4725 | 0/4 |
| 250 | 0/6 | 2800 | 1/9 |
| 80 | 5/20 | 2500 | 0/3 |
| 20 | 11/14 | 1575 | 1/4 |
| | | 800 | 6/12 |
| | | 200 | 9/15 |
| CRU frequency | 1/73,000 | CRU frequency | 1/95,000 |
| (range defined | +1/59,000 | (range defined | +1/75,400 |
| by ± SEM) | −1/91,000 | by ± SEM) | −1/119.000 |

To further investigate the pace and timing of the transition of HSCs into a largely quiescent population, the cycling status of CRUs in lin⁻ BM cell suspensions from 3 and 4 week-old (weanling) mice was analysed.

Table 2 shows data obtained in initial experiments in which the frequencies of CRUs in the lin⁻ BM cells obtained from the 3 and 4 week-old mice were found to be the same (1 per 6.5×10³ and 1 per 6.3×10³ lin⁻ cells) and ˜2-fold lower than in the lin⁻ BM cells from 10 week-old (young adult) mice (1 per 2.9×10³ lin⁻ cells).

TABLE 2
Limiting dilution data for CRU frequency determinations for lin⁻ BM
cells from 3 and 4 week-old mice (pooled data from 2 experiments).

| 3 week BM | | 4 week BM | |
|---|---|---|---|
| | Negative mice/ | | Negative mice/ |
| Cells injected/ | total mice | Cells injected/ | total mice |
| mouse (×10³) | injected | mouse (×10³) | injected |
| 12 | 0/3 | 11 | 0/3 |
| 4 | 4/6 | 4 | 4/6 |
| CRU frequency | 1/6,500 | CRU frequency | 1/6,300 |
| (range defined | +1/4,000 | (range defined by ± | +1/4,000 |
| by ± SEM) | −1/10,000 | SEM) | −1/10,000 |

BM cells from 3 and 4 week-old mice were then fractionated by FACS into their component G₀ and G₁/S/G₂/M subsets based on the gates shown in the left panels of FIG. 3, Part B and Part C, and the sorted G₀ and G₁/S/G₂/M cells were assayed separately for CRU activity. Re-analysis of the sorted G₀ and G₁/S/G₂/M fractions after staining for Ki67 confirmed that the cells expressing this proliferation marker were confined to those we had designated as G₁/S/G₂/M (see representative profiles in the right panels of FIG. 3, Part B). Remarkably, the results of the in vivo assays showed that all of the CRUs detected in the BM of 3 week-old mice were also confined to the G₁/S/G₂/M fraction, whereas >98% of the CRUs in the BM of 4 week-old mice were found in the G₀ fraction (FIG. 4). Thus, there is a rapid down-regulation of CRU proliferative activity in the BM of mice between 3 and 4 weeks of age with little change in CRU numbers.

HSCs in S/G₂/M show a specific and reversible engraftment defect regardless of their developmental origin or route of injection into assay recipients. Given the previously reported engraftment defect of adult HSCs stimulated to enter S/G₂/M,¹⁹ it was of interest to determine whether the number of proliferating HSCs present early in development might be routinely underestimated due an inability of those in S/G₂/M to be detected. To investigate this possibility, the G₁/S/G₂/M population of day 14.5 Ter119⁻ FL cells was subdivided into its component G₁ and S/G₂/M fractions and then each of these 2 subsets was assayed separately for CRUs.

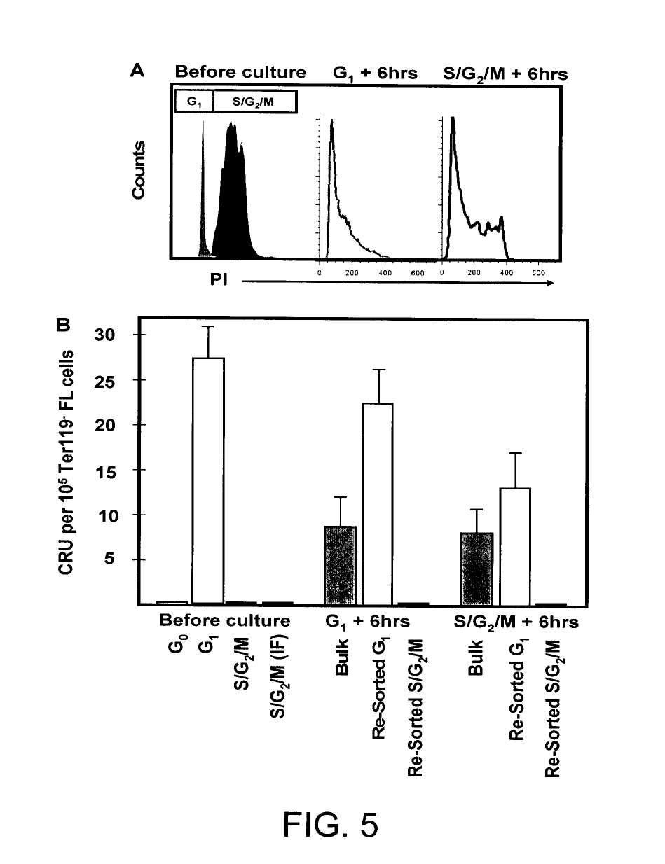

FIG. 5 shows that Hst/Py-sorted HSCs display an absolute but transient S/G₂/M engraftment defect. In Part A, E14.5 Ter119⁻ FL cells in G₁/S/G₂/M were fractionated into their component G₁ and S/G₂/M subsets, leaving a slight separation between them. Aliquots of the sorted subsets were then stained with PI (or Hst/Py, data not shown). The sorted cells were cultured for 6 hours and then were stained again with PI. This showed that, during this 6 hour culture period, approximately a third of the cells originally in G₁ had progressed into S/G₂/M and a similar proportion of the cells originally in S/G₂/M had progressed into G₁. In Part B, CRUs per 10⁵ initial Ter119⁻ FL cells for G₁ and S/G₂/M fractions before and after 6 hours in culture are shown. There was a 3.5-fold loss of CRUs when G₁ cells were cultured for 6 hours (P<0.01), but no loss when the cultured cells were re-sorted for G₁ cells (P=0.36). Conversely, we detected a >65-fold increase (P<0.001) in the number of CRUs detected when CRUs in S/G₂/M were cultured and a >128-fold increase (P<0.001) when the cultured cells were sorted for G₁ cells. Values shown are the mean±SEM of results from at least 3 experiments.

In this case, the gate settings chosen to separate the G₁ (2n DNA) and S-phase cells (>2n DNA) were validated by the profiles obtained when the sorted cells were stained with propidium iodide (PI) and reanalyzed by FACS (FIG. 5, part A, left panel).

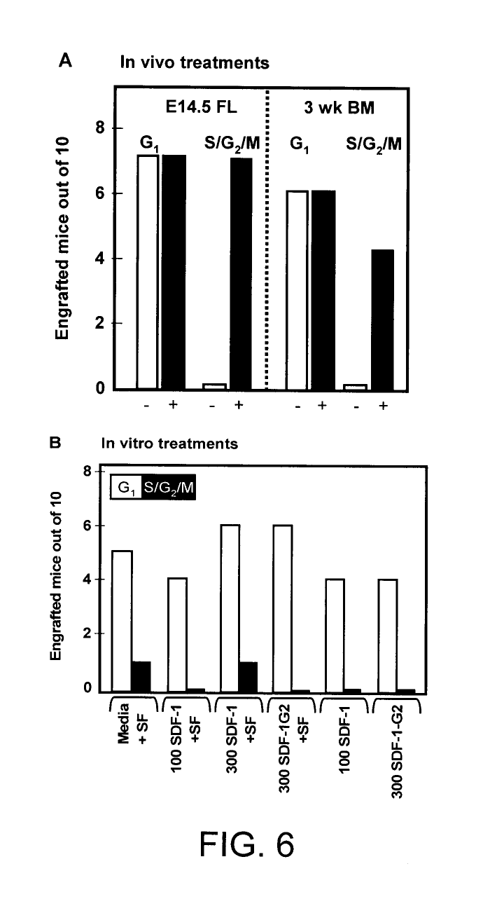

FIG. 6 illustrates that the engraftment defect of HSCs in S/G₂/M is corrected by treatment of the host, but not the cells, with SDF-1G2. In Part A, the effect of injecting prospective recipients is shown at 2 hours post-irradiation and 2 hours prior to transplant with 10 ng/ml SDF-1G2 (+) or PBS (−). Starting equivalents of 4,000 G₁ cells per recipient mouse or 12,000 S/G₂/M cells per recipient mouse were similarly tested. Results show a new ability of FL HSCs in S/G₂/M and 3 week BM HSCs in S/G₂/M to engraft only when they are transplanted into SDF-1G2 treated recipients, whereas treated recipients were no more likely to be engrafted long term by HSCs in G₁ than were untreated recipients. Results are combined from 3 independent experiments. Part B depicts the effect of in vitro treatment of sorted Ter119⁻ FL cells in G₁ or S/G₂/M for 30 minutes at 37° C. in serum-free medium plus various additives, as shown, on CRU detection. When present, SF was used at a concentration of 50 ng/mL, SDF-1 at either 100 ng/ml or 300 ng/ml and SDF-1G2 at 300 ng/ml. In vitro treatment had no significant effect on the number of mice that subsequently showed multi-lineage repopulation from starting cells in either G₁ or S/G₂/M. Results are combined from 3 independent experiments.

All CRU activity detectable in the and G₁/S/G₂(M fraction of Ter119⁻ day 14.5 FL cells was confined to the G₁ subset (left bars in FIG. 5, Part B and control values in the left side of FIG. 6, Part A). Similar experiments performed with lin⁻ BM cells from 3 week-old mice showed that the CRUs in the G₁/S/G₂/M population from this source were likewise confined to the G₁ fraction (control values in the right side of FIG. 6, Part A and data not shown). It is of interest to note that the S/G₂/M defect was specific to repopulating cells able to produce progeny in all lineages for at least 16 weeks. In contrast, cells with short term repopulating activity (8 weeks) were readily detected in the S/G₂/M fraction as well as in the G₁ fraction, thus confirming the restriction of this cell-cycle-dependent engrafting defect to cells with sustained multi-lineage repopulating activity.^{34;35}

To determine whether the apparent engraftment defect of proliferating CRUs was reversible, we first assayed the CRU content of aliquots of the same isolated G₁ and S/G₂/M cells after they had been incubated for 6 hours at 37° C. in serum-free medium containing 50 ng/ml of SF. During this time, many of the G₁ cells progressed into S/G₂/M and many of the S/G₂/M cells moved into G₁, as seen by their altered PI (FIG. 5, part A, right panel) or Hst (data not shown) staining profiles. In vivo assays showed that CRU activity reappeared when the S/G₂/M cells were cultured for 6 hours, whereas the CRU activity originally present in the G₁ cells was partially lost (middle and left bars of FIG. 5, Part B).

Subsequently, it was investigated whether the inability of intravenously transplanted CRUs in S/G₂/M to engraft recipient mice might be overcome by injecting the cells directly into the femoral BM space. However, intrafemoral injection did not enable any CRUs in this subset of Ter119⁻ day 14.5 FL cells to be detected (FIG. 5, part B) even though the frequency of CRUs measured in the corresponding G₁ day 14.5 FL cells after intrafemoral injection was the same as when the latter were transplanted intravenously (1 per 3.6×10³ cells versus 1 per 3.8×10³ cells).

The S/G₂/M engraftment defect of HSCs can be overcome by pretreatment of the host with a SDF-1 antagonist. Previous reports have shown that SDF-1 can promote both the mobilization³⁶ and the homing³⁷⁻³⁹ of HSCs. However, the mobilization of primitive hematopoietic cells can also be stimulated by blocking SDF-1/CXCR4 signaling, as achieved by in vivo administration of AMD3100, a SDF-1 antagonist.⁴⁰ Targeting the SDF-1/CXCR4 pathway may influence the variable engraftment properties of cycling HSCs, by influencing either the HSCs themselves or the transplanted host. The instant example investigates these possibilities, by assessing whether pretreating either the cycling HSCs to be transplanted or their hosts with a specific antagonist of SDF-1 might alter the level of repopulation obtained 16 weeks later. The SDF-1 antagonist used in these experiments was SDF-1G2 (also called P2G because it is identical to SDF-1 except that the proline at position 2 has been converted to glycine⁴²). SDF-1G2 is thus structurally quite different from AMD3100 but similar in its ability to block SDF-1 from binding to CXCR4 without activating CXCR4.^{42;43} SDF-1G2 also shares with AMD3100 an ability to elicit effects on primitive, non-proliferating, hematopoietic cells both in vitro and in vivo.⁴⁴ When mice were injected with 10 μg of SDF-1G2 (or PBS) 2 hours prior to the transplantation of FACS-sorted G₁ or S/G₂/M cells and then analyzed for the presence of donor-derived blood cells 16 weeks later, the results for day 14.5 FL and 3-week mouse BM cells were similar (FIG. 6, Part A). Treatment of recipients with SDF-1G2 had no effect on the repopulating activity of CRUs in G₁. In contrast, and quite unexpectedly, SDF-1G2 pretreatment of recipients of S/G₂/M cells enabled long-term multi-lineage repopulation to be readily detected (7 and 4 of 10 mice transplanted with FL and 3-week BM S/G₂/M cells, respectively, vs. 0 of 10 in both sets of controls injected with PBS, in a total of 3 experiments).

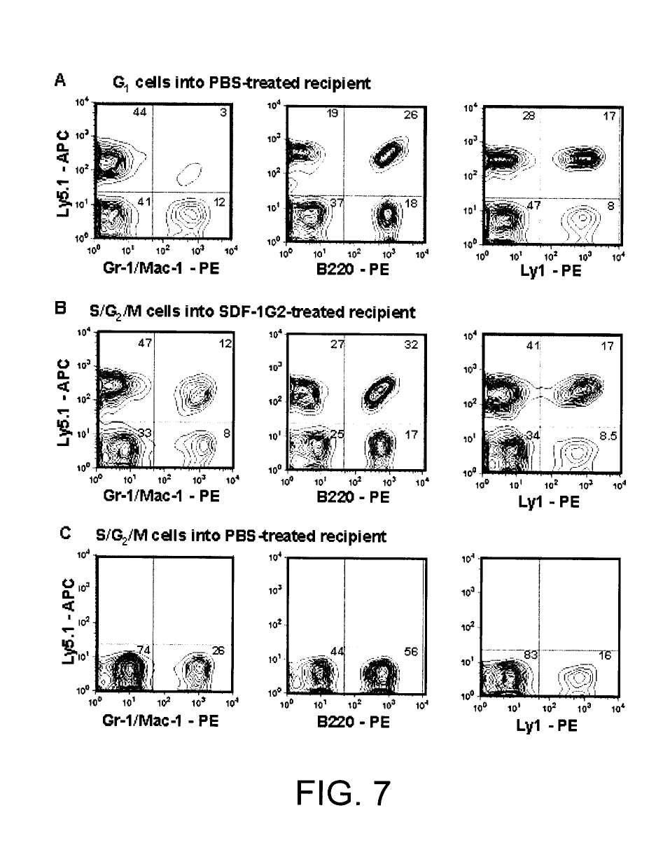

FIG. 7 demonstrates that the SDF-1G2 pretreated hosts showed levels of repopulation by both sources of S/G₂/M cells that were indistinguishable from those seen in mice transplanted with G₁ cells. On the other hand, if the SDF-1G2 treatment was applied directly to the cells to be transplanted for 30 minutes before they were injected, no difference in the engrafting activity of the transplanted G₁ or S/G₂/M cells was seen by comparison to untreated controls over a wide range of SDF-1G2 and SDF-1 concentrations tested, either with or without added SF (FIG. 6, Part B).

FIG. 7 illustrates donor-derived repopulation of SDF-1G2-treated mice. Shown are representative FACS profiles of donor-specific cells detected after dual staining for the donor-type Ly5 allotype and various lineage-specific markers. Part A shows an example of a positively engrafted PBS-treated recipient of FL cells in G₁. Part B shows an example of a positively engrafted SDF-1G2-treated recipient of FL cells in S/G₂/M. Part C shows an example of a PBS-treated recipient of FL cells in S/G₂/M that does not show donor-derived hematopoiesis.

HSCs in S/G₂/M express higher levels of SDF-1 transcripts than HSCs in G₁. To begin to understand the mechanism behind the observed HSC S/G₂/M engraftment defect and how it might be overcome by SDF-1G2 pretreatment of the host, we isolated highly purified populations of HSCs from day 14.5 FLs and from the BM of 3 week-old mice (lin⁻ Sca-1⁺ CD43⁺ Mac1⁺ cells representing ˜20% pure HSCs, and sorted these into their corresponding G₀/G₁ and S/G₂/M fractions as revealed by Hst staining. Aliquots of from ˜200 to 800 cells were collected from each fraction in 3 independent sorting experiments and transcript levels for Gapdh, c-Kit, c-mpl, CD44, α4-integrin (α4int), VCAM-1, CXCR4 and SDF-1 were measured by quantitative real-time analysis of the cDNAs prepared from the isolated RNA extracts, as described in the Methods.

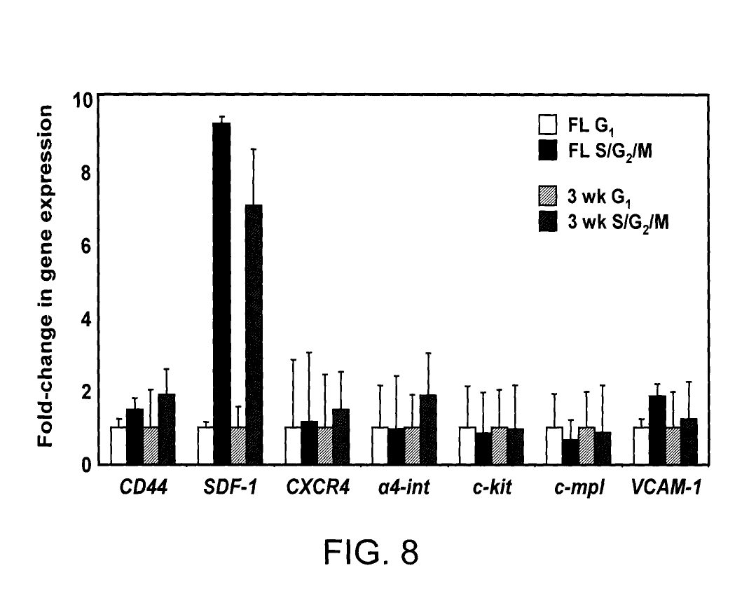

FIG. 8 shows gene expression analysis of the G₁ and S/G₂/M subsets of highly purified lin⁻Sca1⁺CD43⁺Mac1⁺ HSCs from FL and 3-week BM. Gene expression in G₁ was set equal to 1 and the fold change in transcript levels in the corresponding S/G₂/M fraction is shown. Results shown are the mean±SEM of data from 2-3 biological replicates measured in triplicate. The difference between the level of SDF-1 transcripts between the 2 pairs of G₁ and S/G₂/M samples is significant (P<0.05).

Transcripts for all of these genes were consistently detected in both the G₀/G₁ and S/G₂/M fractions of the highly purified suspensions of HSCs from FL and 3 week BM, including SDF-1, which had not previously been shown to be expressed by HSCs (FIG. 8). Interestingly, SDF-1 was also the only one of the genes assessed that was found to be expressed at significantly different levels in G₀/G₁ and S/G₂/M HSCs (9-fold higher in the latter, P<0.05).

Discussion

This example presents 2 new and clinically relevant features of HSC regulation. The first is the unanticipated sudden and complete change in HSC proliferative activity that occurs in juvenile mice between 3 and 4 weeks of age. Both the abruptness and the reproducibility of this change suggest an underlying mechanism that is tightly controlled and broadly active. It is notable that this change was not linked to the migration of HSCs during late embryogenesis from the microenvironment of the FL to that of the BM, but rather, was strictly associated with the developmental status of the donor. Thus, although differences between BM and FL niches and stromal cells have been sought and described,⁴⁵⁻⁴⁸ these differences do not appear to directly determine the cycling activity of the HSCs they are thought to regulate. The present data are more consistent with a model in which the mechanism of HSC cycling control in vivo is indirectly controlled by external cues, perhaps via changing stimulation of the stromal cells that then alter the signals they deliver, as suggested by studies of the long term marrow culture system^{44;49} and of elements of the BM microenvironment in vivo.²⁵ However, internally programmed changes in HSC responsiveness to external factors could also contribute to a developmentally controlled alteration in HSC cycling activity.

In humans, an abrupt change in HSC proliferative activity at an analogous point in development (2-4 years) has been inferred from measurements of the rate of decline in telomere length of circulating leukocytes⁵⁰. This suggests that the mechanisms involved in regulating HSC proliferative activity during ontogeny may be preserved across these species and the mouse will be a relevant model for their future elucidation. It is interesting to note that, in the mouse, a number of other changes in hematopoietic cell properties or output parameters have already been found to change during ontogeny in concert with this transition of the HSC compartment from a predominantly cycling to a predominantly quiescent population. These changes include the initial acquisition of an SP and Rhodull phenotype by HSCs¹⁵, and the completion of appearance and rapid cycling of adult-type (Ly49⁺) natural killer cells and peripheral T-cells.^{51;52}

Many other differences in the properties of fetal and adult HSCs and the programs they dictate have also been described.^{13;53;54} Several genes have been implicated in the differential control of HSC behavior at different stages of development. These include genes encoding various transcription factors, i.e., Runx1⁵⁵, Notch⁵⁶, Scl⁵⁷, bmi1^{58;59} and Gfi-1^{60;61}, as well as the growth factor receptors, c-Kit^{52;63} and Tie2⁶⁴. Further delineation of the molecular basis of the unique programs operative in fetal HSCs and how these regulate fetal HSCs cycling are of major interest as this information could provide new strategies for expanding HSCs and offer potential insights into mechanisms of leukemogenesis.

The second significant set of findings emanating from this example are the universality and pronounced extent of the engraftment defect found to characterize cycling HSCs in the S/G₂/M phases of the cell cycle, the specificity of this effect for hematopoietic cells with prolonged versus short term repopulating activity, and the reversibility of this defect seen either following their progression into G₁ or when the host was pretreated with a specific antagonist of SDF-1. Interestingly, the corrective effect of in vivo administered SDF-1G2 could not be replicated by treatment of the cells with this agent prior to injection. The in vivo effect of SDF-1G2 could also not be mimicked by intrafemoral injection of the test cells. The inability of intrafemoral injection to overcome the defective engraftment of HSCs in S/G₂/M suggests that this defect is likely mediated by events that affect the transplanted HSCs after they have entered the BM environment.

Quantitative analysis of the level of expression of 7 candidate genes in the G₁ and S/G₂/M subsets of purified cycling HSCs from both FL and 3 week BM sources confirmed the expected expression of c-Kit, c-mpl, CD44, α4int, VCAM-1 and CXCR4 and further revealed that these cells also all contain SDF-1 transcripts. Moreover, although the transcript levels were not different between the G₁ and S/G₂/M fractions for c-Kit, c-mpl, CD44, α4int, VCAM-1 and CXCR4, a 9-fold increase in SDF-1 expression was noted in the S/G₂/M HSCs. Previous work has suggested that the ability of transplanted HSCs to reach a niche within the BM that can support their self-maintenance may depend on the strength of the SDF-1 gradient they encounter within the BM space, causing them to migrate towards the osteoblasts that line the bone.²² According to such a model, the ability of HSCs to express SDF-1, themselves, in the absence of changes in their expression of CXCR4, might be anticipated to regulate their ability to respond to other more distal sources of SDF-1. Up-regulated expression of SDF-1 during the progression of HSCs through S/G₂/M, as demonstrated here, might then be sufficient to interfere with an appropriate intra-BM migratory response resulting in the rapid differentiation, death or irreversible sequestration of these cells in a site where they could not be stimulated to divide. Timed blockade of CXCR4 on cells within the BM by injected SDF-1G2 might then be envisaged to increase the level of intra-marrow SDF-1 to a point that transiently restores an effective chemoattractant gradient for the otherwise insensitive HSCs in S/G₂/M. Such a possibility has, in fact, recently been modeled in the zebrafish, where overexpression of SDF-1 in the germ cells was found to prevent the normal migration of these cells towards endogenous SDF-1 signals.⁶⁵

In the hematopoietic system, it is interesting to note that long term repopulating SDF-1−/− HSCs could engraft irradiated hosts whereas only short term repopulation was obtained from CXCR4−/− cells.^{66;67} Forced overexpression of CXCR4 in retrovirally-ransduced (i.e. proliferating) human HSCs was able to enhance the in vivo engrafting activity of these cells⁶⁸ and, conversely, treatment with antibodies to CXCR4 had the opposite effect.⁶⁹ However, SDF-1 levels in the BM are also subject to regulation, for example, as occurs following the administration of granulocyte colony-stimulating factor (G-CSF).⁷⁰

The fact that proliferating human HSCs show the same engraftment defect when they transit S/G₂/M is noteworthy²⁰ and underscores the clinical implications of these findings. For example, the results presented in this example suggest that intrafemoral injection of transplants is unlikely to be a useful strategy for improving the therapeutic effectiveness of HSCs induced to expand in vitro. To date, interference of SDF-1 action by specific CXCR4 inhibitors has been used primarily for enhancing the yield of HSCs from donors for transplantation into myeloablated patients.^{71;72} Another application of such inhibitors suggested by the findings reported here could be to treat recipients of transplants of cycling cells. Thus significant benefit might also be derived by pretreatment of the host, particularly when transplants of genetically modified or cultured cells are to be administered since half of the HSCs in an asynchronously dividing population would be expected to be in S/G₂/M.

The above-described embodiments of the present invention are intended to be examples only. Alterations, modifications and variations may be effected to the particular embodiments by those of skill in the art without departing from the scope of the invention, which is defined solely by the claims appended hereto.

### REFERENCES

1. Szilvassy et al. 1990. Proc. Natl. Acad. Sci. USA. 87:8736-8740.
2. Harrison, D. E et al 1984. J. Exp. Med. 160:759-771.
3. Fleming, W. H. et al. 1993. J. Cell Biol. 122:897-902.
4. Leemhuis, T. et al. 1996. Exp. Hematol. 24:1215-1224.
5. Osawa, M. et al., 1996. Science. 273:242-245.
6. Randall, T. D. et al. 1996. Blood. 87:4057-4067.
7. Bertoncello, I. et al. 1985. Exp. Hematol. 13:999-1006.
8. Spangrude, G. J. et al. 1990. Proc. Natl. Acad. Sci. USA. 87:7433-7437.
9. Wolf, N. S. et al. 1993. Exp. Hematol. 21:614-622.
10. Scharenberg, C. W. et al. 2002. Blood. 99:507-512.
11. Tajima, F. et al. 2001. Blood. 97:2618-2624.
12. Tajima, F. et al. 2000. Blood. 96:1989-1993.
13. Rebel, V. I. et al. 1996. Exp. Hematol. 24:638-648.
14. Morrison, S. J. et al. 1995. Proc. Natl. Acad. Sci. USA. 92:10302-10306.
15. Uchida, N. et al. 2004. Blood. 103:4487-4495.
16. Passegue, E. et al. 2005. J. Exp. Med. 202:1599-1611.
17. Benveniste, P. et al. 2003. Nat. Immunol. 4:708-713.
18. Matsuzaki, Y. et al. 2004. Immunity. 20:87-93.
19. Habibian, H. K. et al., 1998. J. Exp. Med. 188:393-398.
20. Glimm, H. et al. 2000. Blood. 96:4185-4193.
21. Orschell-Traycoff, C. M. et al. 2000. Blood. 96:1380-1387.
22. Lapidot, T. et al. 2005. Blood. 106:1901-1910.
23. Denhardt, D. T. et al. 2001. J. Clin. Invest. 107:1055-1061.
24. Lee, B. C. et al. 2003. Genes Dev. 17:1592-1604.
25. Stier, S. et al., 2005. J. Exp. Med. 201:1781-1791.
26. Bonig, H. et al. 2006. Blood. 107:79-86.
27. Becker, P. S. et al. 1999. Exp. Hematol. 27:533-541.
28. Medvinsky, A. L et al. 1993. Nature. 364:64-66.
29. Gothert, J. R. et al. 2005. Blood. 105:2724-2732.
30. Ponchio, L. et al. 1995. Blood. 86:3314-3321.
31. Uchida, N. et al., 2003. Exp. Hematol. 31:1338-1347.
32. Ladd, A. C. et al. 1997. Blood. 90:658-668.
33. Szilvassy, S. J. et al., 2003. Exp. Hematol. 31:331-338.
34. Szilvassy, S. J. et al. 2000. Blood. 95:2829-2837.
35. Glimm, H. et al. 2001. J. Clin. Invest. 107:199-206.
36. Hattori, K. et al. 2001. Blood. 97:3354-3360.
37. Cashman, J. et al. 2002. J. Exp. Med. 196:1141-1149.
38. Fukuda, S et al. 2005. Blood. 105:3117-3126.
39. Lapidot, T. et al. 2002. Leukemia. 16:1992-2003.
40. De Clercq, E. 2005. Mini. Rev. Med. Chem. 5:805-824.
41. De Clercq et al. 1994. Antimicrob. Agents Chemother 38:668-674.
42. Crump, M. P et al. 1997. EMBO J. 16:6996-7007.
43. De Clercq, 2003. The bicyclam AMD3100 story. Nat. Rev. Drug Discov. 2:581-7.
44. Cashman, J et al. 2002. Blood. 99:792-799.
45. Moore, K. A. 2004. Curr. Opin. Hematol. 11:107-111.
46. Heissig, B. et al., 2005. Hematology. 10:247-253.
47. Lemischka, I. R. 1997. Stem Cells 15 (Suppl 1):63-68.
48. Hackney, J. A. et al. 2002. Proc. Natl. Acad. Sci. USA. 99:13061-13066.
49. Eaves, C. J. et al. 1991. Blood. 78:110-117.
50. Rufer, N. et al. 1999. J. Exp. Med. 190:157-167.
51. Dorfman, J. R. et al., 1998. J. Exp. Med. 187:609-618.
52. Jamieson, A. M et al. 2004. J. Immunol. 172:864-870.
53. Rebel et al. 1996. Blood. 87:3500-7.
54. Pawliuk, R. et al., 1996. Blood. 88:2852-2858.
55. Okuda, T. et al. 2001. Int. J. Hematol. 74:252-257.
56. Hadland, B. K. et al. 2004. Blood. 104:3097-3105.
57. Lecuyer, E. et al. 2004. SCL: Exp. Hematol. 32:11-24.
58. Lessard, J. et al. 2003. Nature. 423:255-260.
59. Park, I. K. et al., 2003. Nature. 423:302-305.
60. Hock, H. et al. 2004. Nature. 431:1002-1007.
61. Zeng, H. et al. 2004. EMBO J. 23:4116-4125.
62. Ikuta et al. 1992. Proc. Natl. Acad. Sci. USA. 89:1502-1506.
63. Miller, C. L. et al. 1997. Blood. 89:1214-1223.
64. Puri, M. C. et al. 2003. Proc. Natl. Acad. Sci. USA. 100:12753-12758.
65. Doitsidou, M. et al., 2002. Cell. 111:647-659.
66. Kawabata, K. et al. 1999. Proc. Natl. Acad. Sci. USA. 96:5663-5667.
67. Ma, Q. et al. 1999. Immunity. 10:463-471.
68. Brenner, S. et al. 2004. Stem Cells. 22:1128-1133.
69. Peled, A. et al., 1999. Science. 283:845-848.
70. Petit, I. et al. 2002. Nat. Immunol. 3:687-694.
71. Broxmeyer, H. E., et al. 2005. J. Exp. Med. 201:1307-1318.
72. Flomenberg, N., et al. 2005. Blood. 106:1867-1874.
73. Randall, T. D et al. 1997. Blood. 89:3596-3606.
74. Mazurier et al. 2003. Nat. Med. 9:959-63.
75. Szilvassy, S. et al. 2002. Methods Mol. Med. 63:167-188.
76. Livak, K. J. et al. 2001. Methods. 25:402-408.
77. Schwarz M K et al., 2002. Nature Reviews Drug Discovery 1, 347-58.
78. Terricabras E et al. 2004. Drug discovery and chemokine receptor antagonists: eppur si muove! Autoimmun Rev 3:550-6.
79. Lataillade J J et al. 2004. Eur Cytokine Netw 15:177-88.
80. Glimm H et al., 2002. Blood 99:3454-7.
81. Perez L E et al. 2004. Exp Hematol 32:300-7.
82. Liles W C et al. 2003. Blood 102:2728-30.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. A composition for transplantation of stem cells comprising S/G₂/M phase stem cells and a SDF-1 antagonist.

20. The composition of claim 19, wherein the SDF-1 antagonist is selected from the group consisting of a peptide, a protein, an antibody, RNAi, an antisense nucleotide, and a small molecule.

21. The composition of claim 19, wherein the SDF-1 antagonist is a peptide or a protein.

22. The composition of claim 21, wherein the SDF-1 antagonist is SDF-1G2.

23. The composition of claim 19, wherein the SDF-1 antagonist is a bicyclam.

24. The composition of claim 23, wherein the SDF-1 antagonist is AMD3100.

25. (canceled)

26. (canceled)

27. (canceled)

28. (canceled)

29. (canceled)

30. (canceled)

31. (canceled)

32. (canceled)

33. A method of improving engraftment of S/G₂/M phase stem cells comprising the step of treating ex vivo expanded stem cells with a SDF-1 antagonist prior to delivery to a recipient.

34. The method of claim 33, wherein the SDF-1 antagonist is selected from the group consisting of a peptide, a protein, an antibody, RNAi, an antisense nucleotide, and a small molecule.

35. The method of claim 33, wherein the stem cells are allogenic or autologous to the recipient.

36. The method of claim 33, wherein the stem cells are hematopoietic stem cells.

37. The method of any claim 33, additionally comprising genetic manipulation of the stem cells ex vivo.

38. The method of claim 33, wherein the SDF-1 antagonist is selected from the group consisting of a peptide of and a protein.

39. The method of claim 38, wherein the SDF-1 antagonist is SDF-1G2.

40. The method of claim 33, wherein the SDF-1 antagonist is a bicyclam.

41. The method of claim 40, wherein the SDF-1 antagonist is AMD3100.

42. A method of improving engraftment upon transplant of S/G₂/M phase stem cells comprising the step of treating a transplant recipient with a SDF-1 antagonist prior to transplantation of ex vivo expanded stem cells.

43. The method of claim 42, wherein the SDF-1 antagonist is selected from the group consisting of a peptide, a protein, an antibody, RNAi, an antisense nucleotide, and a small molecule.

44. The method of claim 42, wherein the stem cells are allogenic or autologous to the recipient.

45. The method of claim 42, wherein the stem cells are hematopoietic stem cells.

46. The method of claim 42, additionally comprising genetic manipulation of the stem cells ex vivo.

47. The method of claim 42, wherein the SDF-1 antagonist is a peptide or a protein.

48. The method of claim 47, wherein the SDF-1 antagonist is SDF-1G2.

49. The method of claim 42, wherein the SDF-1 antagonist is a bicyclam.

50. The method of claim 49, wherein the SDF-1 antagonist is AMD3100.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)


SEQUENCE LISTING (17 sequences)

SEQ ID NO: 1 - protein, 67 aa, artificial
  synthetic
KGVSLSYRCPCRFFESHVARANVKHLKILNTPNCALQIVARLKNNNRQVCIDPKLKWIQEYLEKALN

SEQ ID NO: 2 - DNA, 20 bp, artificial
  synthetic
aggacacaccaggcattcat

SEQ ID NO: 3 - DNA, 20 bp, artificial
  synthetic
cctcagtgtttcgtttggtg

SEQ ID NO: 4 - DNA, 25 bp, artificial
  synthetic
ctttatccggagcaccttggccacc

SEQ ID NO: 5 - DNA, 19 bp, artificial
  synthetic
gtcacagtgcgggaactcc

SEQ ID NO: 6 - DNA, 20 bp, artificial
  synthetic
acaagaggagatccgcaaga

SEQ ID NO: 7 - DNA, 21 bp, artificial
  synthetic
gaagctcagcaaatcatccag

SEQ ID NO: 8 - DNA, 20 bp, artificial
  synthetic
agtggcagcaccagtcatct

SEQ ID NO: 9 - DNA, 19 bp, artificial
  synthetic
gagatggctccagcacctt

SEQ ID NO: 10 - DNA, 20 bp, artificial
  synthetic
cggagtcagaatcctccagt

SEQ ID NO: 11 - DNA, 26 bp, artificial
  synthetic
ctggtcagtctcttatatctggaaaa

SEQ ID NO: 12 - DNA, 20 bp, artificial
  synthetic
aactttggcattgtggaagg

SEQ ID NO: 13 - DNA, 20 bp, artificial
  synthetic
atgcagggatgatgttctgg

SEQ ID NO: 14 - DNA, 20 bp, artificial
  synthetic
gagccaacgtcaagcatctg

SEQ ID NO: 15 - DNA, 20 bp, artificial
  synthetic
cgggtcaatgcacacttgtc

SEQ ID NO: 16 - DNA, 20 bp, artificial
  synthetic
tgattgggagagacaaagca

SEQ ID NO: 17 - DNA, 20 bp, artificial
  synthetic
aacaaccgaatccccaactt